(19) United States
(12) Patent Application Publication
(10) Pub. No.: US 2024/0395414 A1
(43) Pub. Date: Nov. 28, 2024

(54) METHODS AND APPARATUS FOR ESTIMATING WEANING STATUS FOR A MECHANICAL CIRCULATORY SUPPORT DEVICE

(71) Applicant: Abiomed, Inc., Danvers, MA (US)

(72) Inventors: Elise Jortberg, Danvers, MA (US); Christian Moyer, Danvers, MA (US)

(73) Assignee: Abiomed, Inc., Danvers, MA (US)

(21) Appl. No.: 18/634,354

(22) Filed: Apr. 12, 2024

Related U.S. Application Data
(60) Provisional application No. 63/496,335, filed on Apr. 14, 2023.
(60) Provisional application No. 63/585,381, filed on Sep. 26, 2023.

(51) Int. Cl. G16H 50/30; A61M 60/17; A61M 60/585; G16H 10/60

(52) U.S. Cl. CPC G16H 50/30; A61M 60/17; A61M 60/585; G16H 10/60; A61M 2205/04; A61M 2205/103; A61M 2205/3303; A61M 2205/3327; A61M 2205/3331; A61M 2205/3365; A61M 2205/502; A61M 2230/04

(57) ABSTRACT

Methods and apparatus for determining a weaning status for a patient associated with a mechanical circulatory support device are provided. The method includes receiving a set of signals from the mechanical circulatory support device implanted in a heart of a patient, determining, using a computer processor, a set of features based, at least in part, on the set of signals, providing the set of features as input to a machine learning model trained to output a weaning status for the patient, and displaying, on a user interface associated with the mechanical circulatory support device, an indication of the weaning status for the patient output from the machine learning model.

### CROSS REFERENCE TO RELATED APPLICATIONS

This application claims the benefit under 35 U.S.C. § 119 (e) to U.S. Provisional Patent Application No. 63/496,335, filed Apr. 14, 2023, and titled, “METHODS AND APPARATUS FOR ESTIMATING WEANING STATUS FOR A MECHANICAL CIRCULATORY SUPPORT DEVICE,” and claims the benefit under 35 U.S.C. § 119 (e) to U.S. Provisional Patent Application No. 63/585,381, filed Sep. 26, 2023, and titled, “METHODS AND APPARATUS FOR ESTIMATING WEANING STATUS FOR A MECHANICAL CIRCULATORY SUPPORT DEVICE,” the entire contents of each of which is incorporated by reference herein.

### FIELD OF THE INVENTION

This disclosure relates to techniques for determining a weaning status based on signals from a mechanical circulatory support device.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems, provide hemodynamic support and facilitate heart recovery. Some heart pump systems may be percutaneously inserted into the heart and can operate in parallel with the native heart to supplement cardiac output. Examples of such devices include the Impella® family of devices (Abiomed. Inc., Danvers, MA). Such heart pump systems may have sensors that detect blood pressure (or assess differential pressures across membranes) and may monitor motor current, and may use the sensor and motor current readings to help identify pump positioning.

Such pumps can be positioned, for example, in a cardiac chamber, such as the left ventricle, to assist the heart. In this case, the pump may be inserted via a femoral artery by means of a hollow catheter and introduced up to and into the left ventricle of a patient's heart. From this position, the pump inlet may draw in blood and the pump outlet may expel the blood into the aorta. In this manner, the heart's function may be replaced or at least assisted by operation of the pump.

An intravascular pump is typically connected to a respective heart pump controller that controls the pump, such as its motor speed, and collects and displays operational data about the blood pump, such as heart signal level, battery temperature, blood flow rate and plumbing integrity. An exemplary heart pump controller is available from Abiomed, Inc. under the trade name Automated Impella Controller®. The controller raises alarms when operational data values fall beyond predetermined values or ranges, for example if a leak, suction, and/or pump malfunction is detected. The controller may include a video display screen upon which is displayed a graphical user interface configured to display the operational data and/or alarms. The controller may be configured to transmit data associated with the operation of the pump and/or physiological information associated with a patient within whom the pump is inserted to an auxiliary device that enables a healthcare provider to view the information at a location remote from the patient.

### SUMMARY

Percutaneous coronary interventions (PCIs) are minimally invasive procedures that are used to open blocked coronary arteries. Examples of PCIs include balloon angioplasties, angioplasties with a stent, and atherectomies. Some patients undergoing PCI may receive support from a mechanical circulatory support (MCS) device during the procedure. Following PCI, as the patient's native heart function improves and less reliance on the MCS device is needed, the patient may be gradually weaned off of the MCS device with the intent to explant the MCS device. The decision of when and how to de-escalate patient support with the intent to explant an MCS device may be a multi-faceted decision. Currently, no set protocol exists for de-escalation of MCS device support. Rather, the decision to wean the patient off of the MCS device by de-escalating MCS device support is typically observational based on the expert best practices of the healthcare provider providing treatment to the patient.

The systems, devices, and methods described herein relate to a data-driven technique that provides context to the decision on when and how to wean a patient off of MCS device support. For instance, some embodiments of the present disclosure relate to a clinical decision support tool configured to display an indication of a patient's weaning status based, at least in part, on metrics determined from MCS device signals sensed by one or more sensors on the MCS device and a model trained on historical patient cohort data. In some embodiments, the model may be implemented as a machine learning (ML) model trained to output a relative score to indicate a patient's tolerance to reduction in MCS device support.

In one aspect, a computer-implemented method is provided. The computer-implemented method includes receiving a set of signals from a mechanical circulatory support device implanted in a heart of a patient, determining, using a computer processor, a set of features based, at least in part, on the set of signals, providing the set of features as input to a machine learning model trained to output a weaning status for the patient, and displaying, on a user interface associated with the mechanical circulatory support device, an indication of the weaning status for the patient output from the machine learning model.

In another aspect, the set of signals include at least one first signal associated with operation of the mechanical circulatory support device and at least one second signal associated with a physiology of the patient. In another aspect, determining a set of features based, at least in part on the set of signals comprises determining one or more of a contractility feature, a pulsatility feature, or a heart rate feature. In another aspect, the method further includes receiving, via the user interface, an indication to determine the weaning status of the patient, and providing the set of features as input to a machine learning model is performed in response to receiving the indication to determine the weaning status. In another aspect, the method further includes receiving, via the user interface, user input associated with weaning the patient off the mechanical circulatory support device, and retraining the machine learning model based, at least in part, on the user input. In another aspect, the method further includes receiving from an electronic medical record associated with the patient, medical information, and providing the medical information as input to the machine learning model. In another aspect, the mechanical circulatory support device includes a heart pump, and the method further includes receiving, via the user interface, an instruction to reduce a speed of the heart pump, sending an instruction to a controller of the heart pump to reduce the speed of the heart pump after receiving the instruction to reduce the speed, and updating the weaning status for the patient after reducing the speed of the heart pump. In another aspect, updating the weaning status for the patient includes determining, using the computer processor, a second set of features based, at least in part, on the set of signals received after reducing the speed of the heart pump, providing the second set of features as input to the machine learning model, and displaying, on the user interface associated with the mechanical circulatory support device, an indication of an updated weaning status for the patient output from the machine learning model when provided with the second set of features as input. In another aspect, displaying, on a user interface associated with the mechanical circulatory support device, an indication of the weaning status for the patient output from the machine learning model comprises displaying a hemodynamic stability score for the patient. In another aspect, the method further includes displaying, on the user interface, one or more user interface elements that enable a user to simulate weaning of the patient off the mechanical circulatory support device, performing a weaning simulation in response to receiving user input via the one or more user interface elements, and displaying, on the user interface, an updated weaning status score determined based on performing the weaning simulation.

In one aspect, a controller for a mechanical circulatory support device is provided. The controller includes at least one hardware processor configured to determine a set of features based, at least in part, on a set of signals received from a mechanical circulatory support device implanted in a heart of a patient, provide the set of features as input to a machine learning model trained to output a weaning status for the patient, and display, on a user interface associated with the mechanical circulatory support device, an indication of the weaning status for the patient output from the machine learning model.

In another aspect, the set of signals include at least one first signal associated with operation of the mechanical circulatory support device and at least one second signal associated with a physiology of the patient. In another aspect, the set of features includes one or more of a contractility feature, a pulsatility feature, or a heart rate feature. In another aspect, the at least one hardware processor is further configured to receive, via the user interface, an indication to determine the weaning status of the patient, and providing the set of features as input to a machine learning model is performed in response to receiving the indication to determine the weaning status. In another aspect, the at least one hardware processor is further configured to receive, via the user interface, user input associated with weaning the patient off the mechanical circulatory support device, and retrain the machine learning model based, at least in part, on the user input. In another aspect, the at least one hardware processor is further configured to receive from an electronic medical record associated with the patient, medical information, and provide the medical information as input to the machine learning model. In another aspect, the mechanical circulatory support device includes a heart pump, and the at least one hardware processor is further configured to receive, via the user interface, an instruction to reduce a speed of the heart pump, reduce the speed of the heart pump after receiving the instruction to reduce the speed, and update the weaning status for the patient after reducing the speed of the heart pump. In another aspect, updating the weaning status for the patient includes determining a second set of features based, at least in part, on the set of signals received after reducing the speed of the heart pump, providing the second set of features as input to the machine learning model, and displaying, on the user interface associated with the mechanical circulatory support device, an indication of an updated weaning status for the patient output from the machine learning model when provided with the second set of features as input. In another aspect, displaying, on a user interface associated with the mechanical circulatory support device, an indication of the weaning status for the patient output from the machine learning model comprises displaying a hemodynamic stability score for the patient. In another aspect, the at least one hardware processor is further configured to display, on the user interface, one or more user interface elements that enable a user to simulate weaning of the patient off the mechanical circulatory support device, perform a weaning simulation in response to receiving user input via the one or more user interface elements, and display, on the user interface, an updated weaning status score determined based on performing the weaning simulation.

In one aspect, a heart pump system is provided. The heart pump system includes a heart pump including at least one pressure sensor configured to sense a pressure within a portion of a heart of a patient and a controller. The controller is configured to determine a set of features based, at least in part, on a set of signals received from heart pump, the set of features a first feature based on the sensed pressure, provide the set of features as input to a machine learning model trained to output a weaning status for the patient, and display, on a user interface associated with the heart pump system, an indication of the weaning status for the patient output from the machine learning model.

In another aspect, the set of signals include at least one first signal associated with operation of the heart pump and at least one second signal associated with a physiology of the patient. In another aspect, the set of features includes one or more of a contractility feature, a pulsatility feature, or a heart rate feature. In another aspect, the controller is further configured to receive, via the user interface, an indication to determine the weaning status of the patient, and providing the set of features as input to a machine learning model is performed in response to receiving the indication to determine the weaning status. In another aspect, the controller is further configured to receive, via the user interface, user input associated with weaning the patient off the mechanical circulatory support device, and retrain the machine learning model based, at least in part, on the user input. In another aspect, the controller is further configured to receive from an electronic medical record associated with the patient, medical information, and provide the medical information as input to the machine learning model. In another aspect, the controller is further configured to receive, via the user interface, an instruction to reduce a speed of the heart pump, reduce the speed of the heart pump after receiving the instruction to reduce the speed, and update the weaning status for the patient after reducing the speed of the heart pump. In another aspect, updating the weaning status for the patient includes determining a second set of features based, at least in part, on the set of signals received after reducing the speed of the heart pump, providing the second set of features as input to the machine learning model, and displaying, on the user interface, an indication of an updated weaning status for the patient output from the machine learning model when provided with the second set of features as input. In another aspect, displaying, on a user interface, an indication of the weaning status for the patient output from the machine learning model comprises displaying a hemodynamic stability score for the patient. In another aspect, the controller is further configured to display, on the user interface, one or more user interface elements that enable a user to simulate weaning of the patient off the mechanical circulatory support device, perform a weaning simulation in response to receiving user input via the one or more user interface elements, and display, on the user interface, an updated weaning status score determined based on performing the weaning simulation.

In one aspect, a computer-implemented method of training a machine learning model used to determine a weaning status for a patient is provided. The computer-implemented method includes receiving historical patient cohort data for a plurality of patients, each of which had an implanted mechanical circulatory support device, the historical patient cohort data including, for each of the plurality of patients, a set of signals associated with the mechanical circulatory support device, associating a label with each of the plurality of patients in the historical patient cohort data, the label indicating whether the patient tolerated weaning off the mechanical circulatory support device or did not tolerate weaning off the mechanical circulatory support device, and training a machine learning model based, at least in part, on the historical patient cohort data and the labels associated with the plurality of patients.

### BRIEF DESCRIPTION OF DRAWINGS

FIG. 1A shows an illustrative cardiac support device that may be used with some embodiments of the present disclosure.

FIG. 1B shows an illustrative cardiac support system that includes the cardiac support device of FIG. 1A.

FIG. 2 illustrates a flowchart of a process for training a model to output a weaning status of a patient, in accordance with some embodiments of the present technology.

FIG. 3A schematically illustrates example features that may be used to train a model to predict a weaning status of a patient, in accordance with some embodiments of the present technology.

FIG. 3B schematically illustrates a patient timeline associated with a PCI procedure for a patient that tolerated weaning and a patient that did not tolerate weaning, in accordance with some embodiments of the present technology.

FIG. 4A schematically illustrates different scenarios for classifying historical patient cohort data, in accordance with some embodiments of the present technology.

FIG. 4B illustrates an example decision tree for determining labels for historical patient cohort data used to train a model, in accordance with some embodiments of the present technology.

FIG. 4C illustrates an alternate example decision tree for determining labels for historical patient cohort data used to train a model, in accordance with some embodiments of the present technology.

FIG. 5 illustrates a flowchart of a process for using a trained model to determine a weaning status for a patient, in accordance with some embodiments of the present technology.

FIG. 6 illustrates a portion of a user interface for initiating a weaning status determination process, in accordance with some embodiments of the present technology.

FIG. 7 illustrates a portion of an alternate user interface for initiating a weaning status determination process, in accordance with some embodiments of the present technology.

FIG. 8 illustrates a portion of a user interface showing calculation of a weaning status determination process in progress, in accordance with some embodiments of the present technology.

FIG. 9 illustrates a portion of a user interface configured to display an indication of a weaning status for a patient, in accordance with some embodiments of the present technology.

FIG. 10 illustrates a portion of an alternate user interface configured to display an indication of a weaning status for a patient, in accordance with some embodiments of the present technology.

FIG. 11 schematically illustrates tracking a weaning status for a patient over time, in accordance with some embodiments of the present technology.

### DETAILED DESCRIPTION

Determining when and how to wean a patient off of support provided by a mechanical circulatory support (MCS) device following an MCS-supported PCI procedure may be a multi-faceted decision that is typically informed solely based on the observation and expertise of the healthcare provider treating the patient. The inventors have recognized and appreciated that both patients and healthcare providers may benefit from a data-driven technique that provides context to the weaning decision process by predicting the hemodynamic stability of a patient. For instance, based on observation alone it may be challenging to quantify acute risk of not weaning a patient from MCS device support or of weaning the patient from MCS device support too early. In the case of weaning too early, such patients may be at higher risk of decompensation or re-implant of the MCS device within a short time (e.g., 48 hours) following device explant. Additionally, there may be large variability across healthcare providers on when and how to wean a MCS-support patient following a PCI procedure. This variability may be exacerbated based on the experience level of the healthcare provider, which may play a large role in determining whether, when, and how to wean the patient. To this end, some embodiments of the present technology may enable healthcare providers of different experience levels to leverage the collective expertise of a large number of healthcare providers and patients who have successfully or unsuccessfully weaned from an MCS device following a PCI procedure.

In some embodiments, data collected from a cohort of patients who underwent MCS-supported PCI procedures and were weaned (successfully or unsuccessfully) off of the MCS device following the PCI procedure may be used to train a predictive model (e.g., a machine learning model). Through training the model may learn characteristics in the input data that are most predictive of whether a patient is likely to successfully wean from an MCS device. For example, the model may apply larger weights to those characteristics through the learning process. Following training, the trained model may be used to guide a weaning process for a patient based on current data associated with the patient as recorded by an implanted MCS device. For instance, the trained model may be trained to output a weaning status score that represents the predicted tolerance of the patient to reduced support from the MCS device.

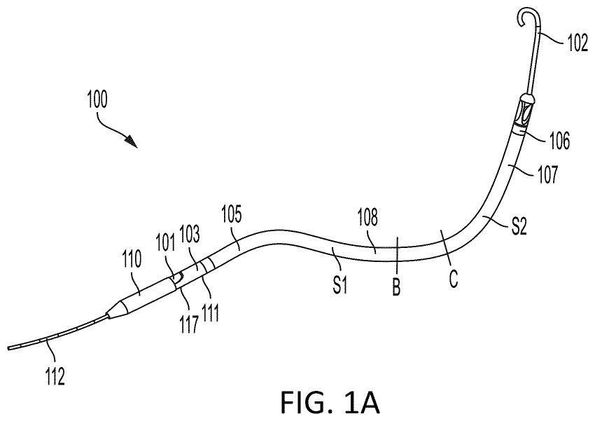

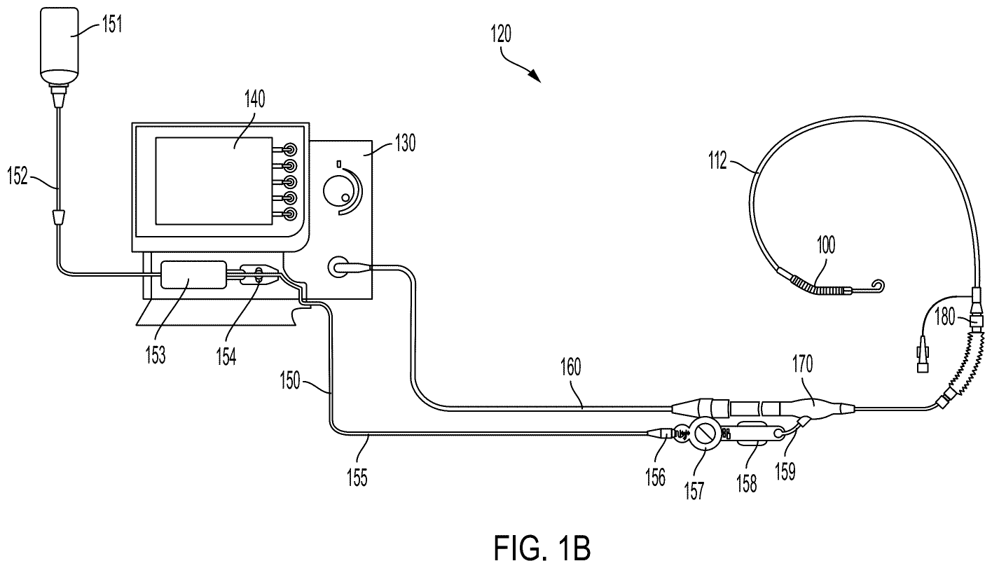

FIG. 1A shows an illustrative embodiment of a blood pump assembly 100 according to the present disclosure. The blood pump assembly 100 may include a pump 101, a pump housing 103, a proximal end 105, a distal end 107, a cannula 108, an impeller (not shown), an atraumatic extension 102, a catheter 112, an inlet area 110, an outlet area 106, and blood exhaust apertures 117. The catheter 112 may be connected to the inlet area 110 of the cannula 108 in some embodiments. The inlet area 110 may be located near the proximal end 105 of the cannula, and the outlet area 106 may be located toward the distal end 107 of the cannula 108. The inlet area 110 may include a pump housing 103 with a peripheral wall 111 extending about a rotation axis of the impeller blades, positioned radially outward of the inner surface with respect to the rotation axis of the impeller. The impeller may be rotatably coupled to the pump 101 at the inlet area 110 adjacent to the blood exhaust apertures 117 formed in the wall 111 of the pump housing 103. The pump housing 103 may be composed of a metal in accordance with some implementations. The extension 102, also referred to as a “pigtail,” may be connected to the distal end 107 of the cannula 108 and may assist with stabilizing and/or positioning the blood pump assembly 100 into the correct position in the heart. The pigtail 102 may be configurable from a straight to a partially curved configuration. The pigtail 102 may be composed, at least in part of a flexible material, and may have dual stiffness. It should be appreciated that some embodiments of the pump assembly may not include pigtail 102.

The cannula 108 may have a shape which matches (or is similar to) the anatomy of the right ventricle of a patient. In the exemplary embodiment shown in FIG. 1A, the cannula has a proximal end 105 arranged to be located near the patient's inferior vena cava, and a distal end 107 arranged to be located near the pulmonary artery. The cannula 108 may include a first segment S1 extending from the inflow area to a point B between the inlet area 110 and the outlet area 106. The cannula 108 may also include a second segment S2 extending from a point C, which is between the inlet area 110 and the outlet area 106, to the outlet area 106. In some implementations, points B and C may be located at the same location along cannula 108. The first segment S1 of the cannula may form an ‘S’ shape in a first plane. In some implementations, segment S1 can have curvatures between 30 degrees and 180 degrees. The second segment S2 of the cannula may form an ‘S’ shape in a second plane. In some implementations, segment S2 can have curvatures between 30 degrees and 180 degrees (e.g., 40°, 50°, 60°, 70°, 80°, 90°, 100°, 110°, 120°, 130°, 140°, 150°, 160°, or) 170°. The second plane can be different from the first plane. In some implementations, the second plane may be parallel or identical to the first plane.

Although shown with an ‘S’ shape, it will be appreciated that other implementations of the blood pump assembly may be formed with other shapes (e.g., a ‘U’ shape), or with no shape at all when outside the body. In such implementations, the cannula may be formed of a flexible material such that the cannula may bend during insertion and achieved the desired shape once inside the heart of the patient.

In some implementations, the blood pump assembly 100 may be inserted percutaneously through the internal jugular vein, though the right atrium and into the right ventricle. When properly positioned, the blood pump assembly 100 may deliver blood from the inlet area 110, which sits inside the patient's right atrium, through the cannula 108, to the blood exhaust apertures 117 of the pump housing 103 positioned in the pulmonary artery. Alternatively, in some implementations the blood pump assembly 100 may be inserted percutaneously through the femoral artery and into the left ventricle to deliver blood from the left ventricle into the aorta.

FIG. 1B shows that blood pump assembly 100 may form part of a cardiac support system 120. Cardiac support system 120 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of blood pump assembly 100. During operation, purge subsystem 150 may be configured to deliver a purge fluid to blood pump assembly 100 through catheter 112 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between blood pump assembly 100 and controller 130. Plug 170 may connect catheter 112, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition blood pump assembly 100 in the patient's heart (e.g., by holding a position of the pump assembly relative to the patient).

As shown in FIG. 1B, in some embodiments, the cardiac support system 120 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 120 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to blood pump assembly 100. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 may include a flexible rubber diaphragm that provides the additional filling volume by means of an expansion chamber. Infusion filter 158 may help prevent bacterial contamination and air from entering catheter 112. Sidearm 159 may provide a fluidic connection between infusion filter 158 and plug 170. Although shown as having separate purge tubing and connector cable, it will be appreciated that in some embodiments, the cardiac support system 120 may include a single connector with both fluidic and electric lines connectable to the controller 130.

During operation, controller 130 may be configured to receive measurements from one or more pressure sensors (not shown) included as a portion of blood pump assembly 100 and purge disc 154. Controller 130 may also be configured to control operation of the motor (not shown) of the blood pump assembly 100 and purge cassette 153. In some embodiments, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter 112 and plug 170. Sensor cables (not shown) within catheter 112, connector cable 160, and plug 170 may provide an electrical connection between components of the blood pump assembly 100 (e.g., one or more pressure sensors) and controller 130. Motor cables (not shown) within catheter 112, connector cable 160, and plug 170 may provide an electrical connection between the motor of the blood pump assembly 100 and controller 130. During operation, controller 130 may be configured to receive measurements from one or more pressure sensors of the blood pump assembly 100 through the sensor cables (e.g., optical fibers) and to control the electrical power delivered to the motor of the blood pump assembly 100 through the motor cables. By controlling the power delivered to the motor of the blood pump assembly 100, controller 130 may be operable to control the speed of the motor.

Various modifications can be made to cardiac support system 120 and one or more of its components. For instance, one or more additional sensors may be added to blood pump assembly 100. In another example, a signal generator may be added to blood pump assembly 100 to generate a signal indicative of the rotational speed of the motor of the blood pump assembly 100. As another example, one or more components of cardiac support system 120 may be separated. For instance, display 140 may be incorporated into another device in communication with controller 130 (e.g., wirelessly or through one or more electrical cables).

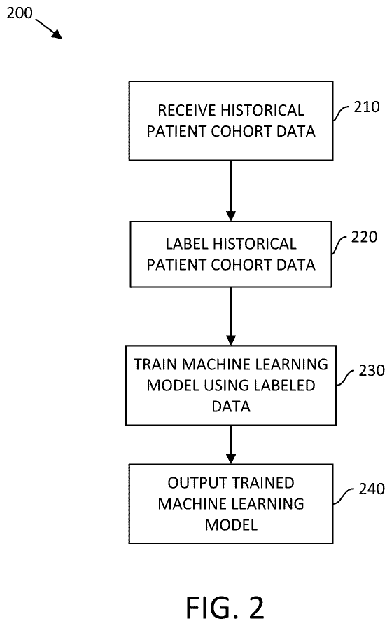

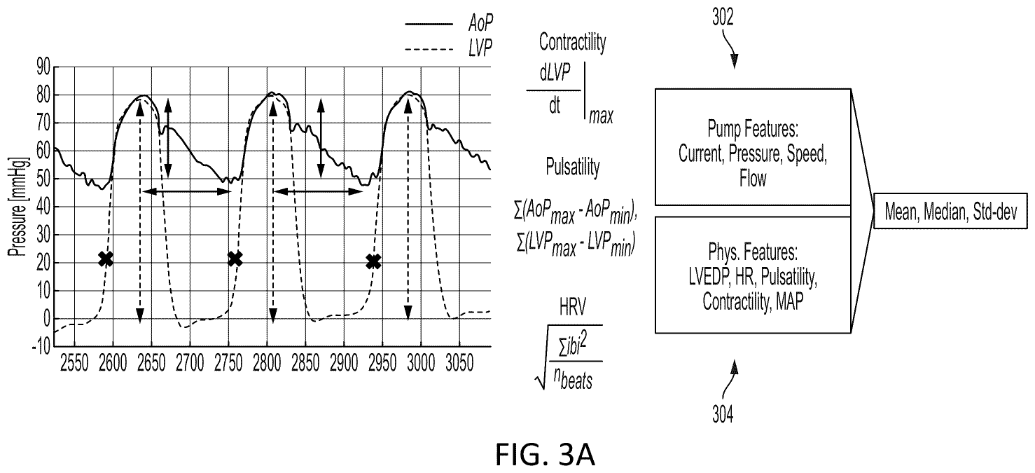

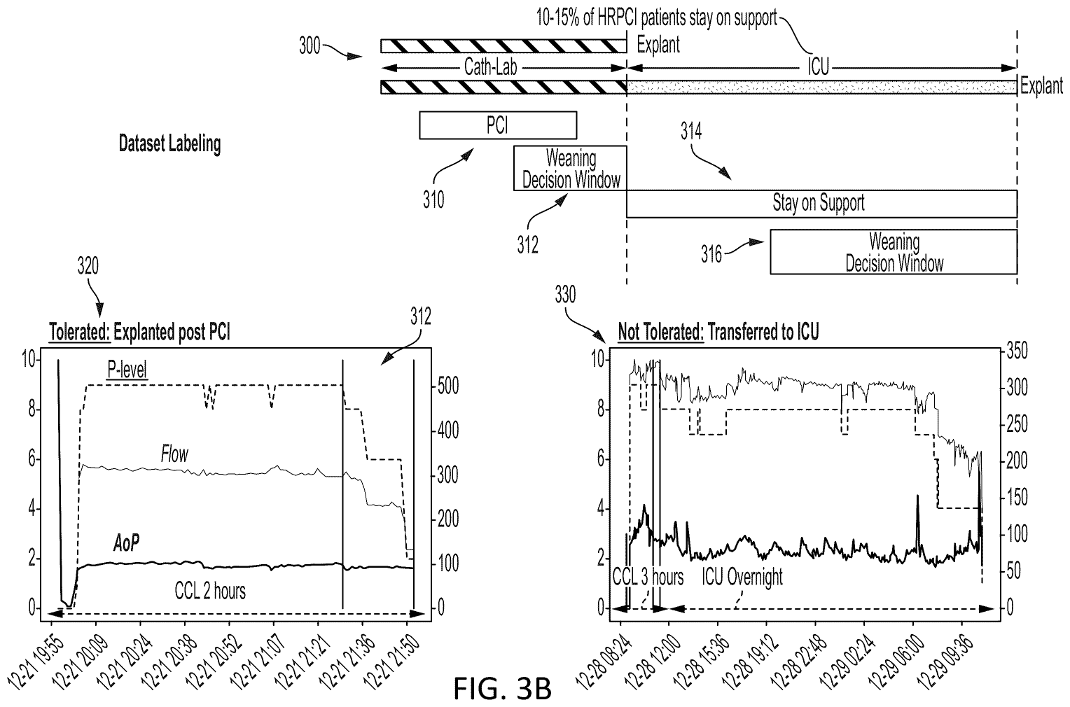

FIG. 2 illustrates a flowchart of a process 200 for training a model to output a prediction of a patient's tolerance to reduced MCS device support, in accordance with some embodiments of the present technology. Process 200 begins in act 210, where historical patient cohort data is received for a plurality of patients that have undergone MCS-device supported PCI procedures (also referred to herein as “high-risk PCI” or “HRPCI” procedures). In some embodiments, the historical patient cohort data may include values for one or more features captured during a weaning time window during weaning of the patient following a PCI procedure. Examples of features that may be included in the historical patient cohort data are shown in FIG. 3A. For example, such features may include, but are not limited to, heart pump operation features 302 (e.g., motor current, pressure information, pump speed, blood flow) and patient physiological features 304 (e.g., left ventricular end diastolic pressure (LVEDP), heart rate, pulsatility, contractility, mean arterial pressure (MAP)). In some embodiments, one or more of the features may be dependent on a motor speed (e.g., P-level) of the heart pump. For instance, a contractility feature may include a ratio of contractility at a high P-level compared to a low P-level, contractility normalized to a particular amount of support provided by the pump (e.g., normalized based on P-level), etc. In some embodiments, other features (e.g., features derived from the patient's electronic health record) may also be included in the historical patient cohort data.

An example patient treatment timeline 300 for a patient implanted with an MCS device is shown in FIG. 3B. As shown in timeline 300, the patient may undergo an MCS-supported PCI procedure during a first time window 310. After the PCI procedure, the patient may enter a weaning decision time window 312, during which it is determined whether to wean the patient off of the MCS device and if so, to initiate weaning by reducing support provided by the MCS device (e.g., by reducing pump speed). Plot 320 shows feature values over time for a patient that tolerated weaning off of the MCS device following the PCI procedure. For example, as shown in plot 320, as the pump speed (P-level), and consequently the blood flow through the MCS device, is decreased during the weaning decision time window 312, the aortic pressure (AoP) measured from the heart of the patient maintains a steady value indicating tolerance of the weaning process. As shown in timeline 300, some patients may not tolerate the weaning process well, and as a consequence the patient may stay on support from the MCS device during time interval 314. After at least some additional MCS device support is provided, another weaning decision time window 316 may occur during which weaning may be attempted. Plot 330 shows feature values over time for a patient that did not tolerate weaning off of the MCS device following the PCI procedure. For example, as shown in plot 330, as the pump speed (P-level) is decreased during a first weaning decision time window, the aortic pressure of the patient is not stable, resulting in the patient being transferred to the intensive care unit with continued support being provided from the MCS device.

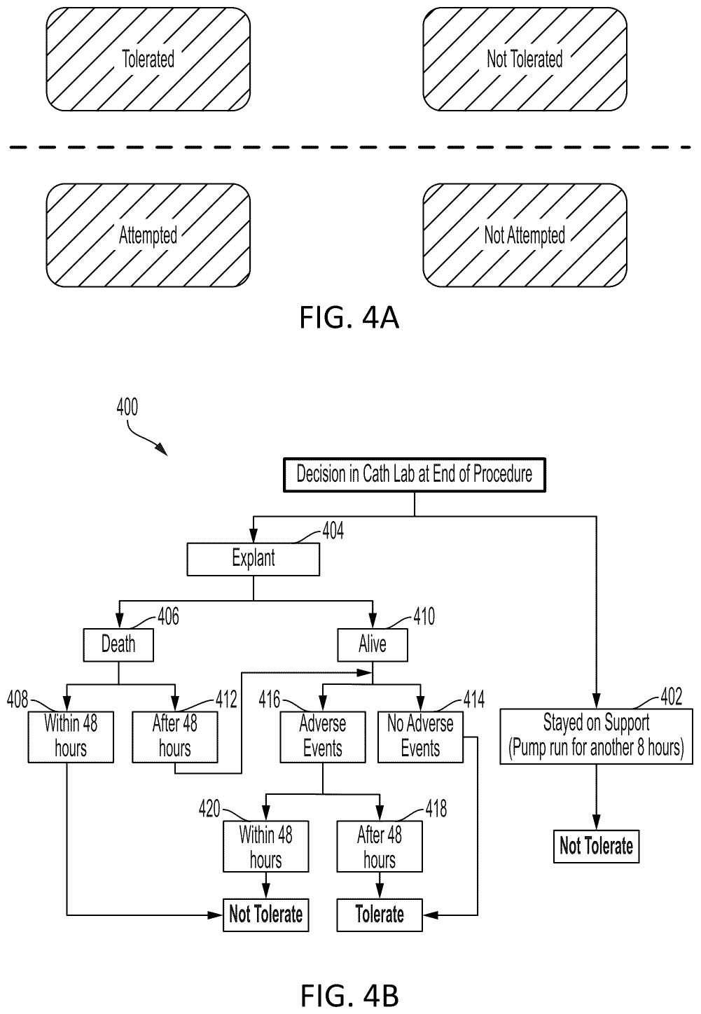

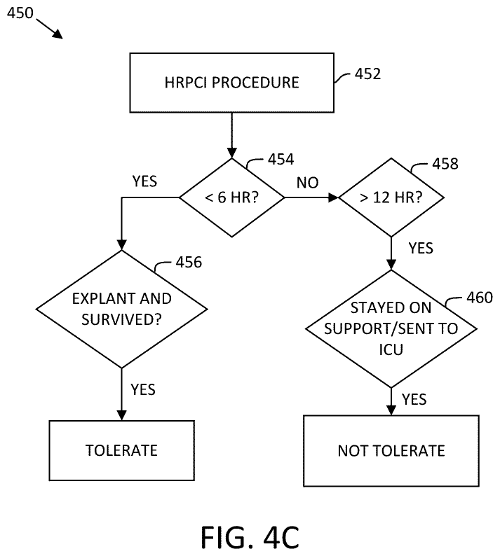

Returning to process 200 shown in FIG. 2, after historical patient cohort data is received in act 210, process 200 may proceed to act 220, where the received historical data is labeled. As shown in FIG. 4A, in some embodiments the received historical data may be labeled according to four different scenarios. In a first scenario, weaning was attempted and was tolerated by the patient. An example of the first scenario is depicted in plot 320 of FIG. 3B. In a second scenario, a typical weaning process of stepping down the pump speed was not attempted, but nevertheless explant of the MCS device was successful. In this scenario, the pump speed may have been decreased to a minimum pump speed (e.g., PO) almost immediately (e.g., during the weaning decision time window 312 shown in timeline 300 of FIG. 3B) from a higher support level without a series of pump speed step downs, which is typical of weaning. In a third scenario, weaning was attempted and was not tolerated by the patient. For instance, step downs in pump speed may have been attempted during weaning, but the patient may have been returned to a higher support level and transferred to the ICU (or may not have met other criteria, examples of which are described below). An example of this scenario is depicted in plot 330 of FIG. 3B. In a fourth scenario, weaning was neither attempted nor tolerated by the patient. In this scenario, the patient may have been sent straight to the ICU following the PCI procedure with no pump speed level reductions at the end of the PCI procedure (e.g., during the weaning decision time window 312 shown in timeline 300 of FIG. 3B).

FIG. 4B illustrates an example of a decision tree 400 that may be used in some embodiments to determine whether a patient having data in the received historical patient cohort data tolerated or did not tolerate reduced MCS device support. As shown in act 402 of FIG. 4B, at the end of the PCI procedure explant may not be attempted for some patients and they may continue to receive MCS support (e.g., fourth scenario above). Those patients may be labeled as not tolerating reduced MCS device support. For patients in which explant is attempted following the PCI procedure (act 404), it may be determined whether the patient died or stayed alive. If the patient died (act 406) within a particular time (e.g., 48 hours) following explant (act 408), the patient may be labeled as not tolerating reduced MCS device support. If the patient stayed alive (act 410) or died after the particular time (e.g., 48 hours) following explant (act 412), it may be determined whether the patient experienced any adverse events. If the patient did not experience any adverse events (act 414), the patient may be labeled as tolerating reduced MCS device support. If the patient experienced one or more adverse events (act 416), it may be determined whether the adverse events were experienced within a particular time (e.g., 48 hours) following explant. If the patient experienced the adverse event(s) more than the particular time (e.g., 48 hours) post explant (act 418), the patient may be labeled as tolerating reduced MCS device support. Otherwise (act 420), the patient may be labeled as not tolerating reduced MCS device support.

FIG. 4C illustrates an example of another decision tree 450 that may be used in some embodiments to determine whether a patient having data in the received historical patient cohort data tolerated or did not tolerate reduced MCS device support. As shown in act 452 of FIG. 4C, an MCS device may be implanted in a patient and a high-risk PCI procedure may be performed. It may then be determined in act 454 whether MCS device support lasted less than a first threshold amount (e.g., 6 hours). If it is determined that the MCS device support lasted less than the first threshold amount, it may be determined in act 456 whether the MCS device was successfully explanted (e.g., in the cardiac catheterization lab) and the patient survived. If it is determined that the MCS device was explanted and the patient survived, the patient may be labeled as tolerating reduced MCS device support. If it is determined in act 454 that the duration of MCS device support exceeded the first threshold amount, it may be determined in act 458 whether the duration of MCS device support exceeded a second threshold amount (e.g., 12 hours). If it is determined that the duration of MCS device support exceeded the second threshold amount, it may be determined in act 460 whether the patient stayed on MCS device support and/or was sent to the intensive care unit. If the patient stayed on MCS device support and/or was sent to the intensive care unit, the patient may be labeled as not tolerating reduced MCS device support. Although not shown in FIG. 4C, decision tree 450 may also include acts that take into consideration the occurrence of adverse events post-explant of a heart pump (e.g., occurring within a particular amount of time post-explant), examples of which are described in connection with decision tree 400 shown in FIG. 4B.

It should be appreciated that decision tree 400 shown in FIG. 4B and decision tree 450 shown in FIG. 4C are illustrative and different labeling schema using different inclusion and/or exclusion criteria may be used in some embodiments to label patients in historical patient cohort data as having tolerated or not tolerated reduced MCS device support.

Returning to process 200 shown in FIG. 2, after each of the patients included in the historical patient data is labeled as tolerating/not tolerating reduced MCS device support in act 220, process 200 may proceed to act 230, where a model (e.g., a machine learning model) is trained using the labeled data. For instance, values for a plurality of features extracted at particular time intervals (e.g., 5 second intervals, 15 second intervals, 30 second intervals) during a weaning period for each patient in the historical patient cohort may be provided as input to the model, and the model may be trained to output the associated label (e.g., tolerated/not tolerated) for the patient. The values for the features may be values at a single point in time, median or mean values determined over a particular time window, and/or measures of variability (e.g., standard deviation) of the values for a particular time window.

In some embodiments, the model may be trained (or retrained) based, at least in part, on input provided by a healthcare provider. For instance, after receiving an indication of a weaning status for a patient using one or more of the techniques described herein, a healthcare provider may provide feedback based on the received indication and/or information associated with the patient that was used to generate the indication of weaning status (e.g., information from the patient's EMR, trend data measured or estimated from the heart pump, etc.). The feedback provided by the healthcare provider may be used to train the model, which may in turn refine the weaning status indications provided by the model in further iterations of the weaning status determination process. In some embodiments, the feedback from the healthcare provider may be provided during the initial training of the model. For instance, a healthcare provider may review data in the historical patient cohort and the weaning status provided as output of the model and provide feedback (e.g., via a user interface) that may be used to further train the model. In this way, some embodiments may provide physician-informed weaning status recommendations with clinically relevant context.

When trained on a large amount of data included in the historical patient cohort, the model may learn (e.g., by changing weights in the model) characteristics of the feature values that predict the corresponding labels for the patients from the labeled data. In some embodiments, the model is a classification model. It should be appreciated that in such embodiments, any suitable classification model may be used, examples, of which include a neural-network (e.g., deep learning) based model, a random forest classifier model, a decision trees model (e.g., a gradient boosting decision trees model), or a logistic regression model. Process 200 then proceeds to act 240, where the trained model is output for use in predicting a weaning status score for patients not included in the historical patient cohort data.

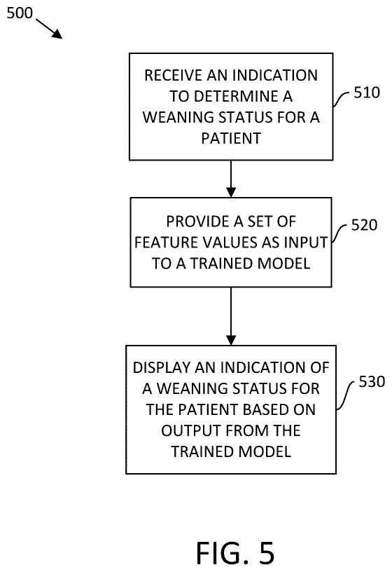

FIG. 5 is a flowchart of a process 500 for determining a weaning status associated with a patient having an MCS device implanted. Process 500 begins in act 510, where an indication to determine a weaning status for a patient is received. It should be appreciated that a determination of a weaning status for a patient using one or more of the techniques described herein may be performed at any suitable time. For instance, in some embodiments, the weaning status may be determined during a PCI procedure and prior to weaning, to determine when to start the weaning process (e.g., based on whether the patient in their current state is likely to tolerate reductions in pump speed or not). In other embodiments, the weaning status may be determined during the weaning process to evaluate how the patient is tolerating the pump speed reductions during weaning. When evaluated during weaning, the weaning status of the patient may be used, for example, to guide the timing of further pump speed reductions and/or pump speed increases, as the patient's weaning status is monitored.

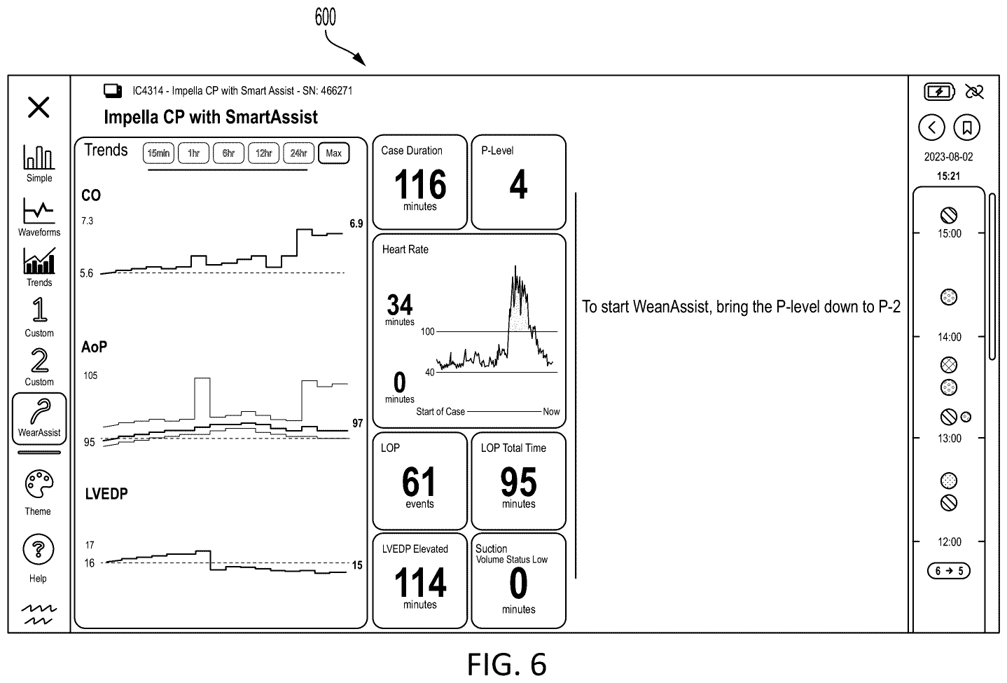

FIG. 6 illustrates an example of a portion of a user interface 600, with which a user (e.g., a healthcare provider) may interact to initiate a weaning status determination process. For instance, information associated with pump operation and physiological parameters associated with the patient within whom an MCS device is implanted may be displayed on the user interface. In the example user interface 600, the display includes different widgets for displaying different types of data including the current pump speed (e.g. P-level), the patient's heart rate, and other metrics including the number and/or total duration of low-volume suction events, the number and/or duration of loss of pulsatility (LOP) events (e.g., any area where the pulse pressure falls below a certain pressure threshold (e.g., 20 mm Hg)), and the duration of elevated left ventricular end diastolic pressure (LVEDP). Some widgets may be configured to display trend information for various metrics over time, such as cardiac output (CO), aortic pressure (AoP), and LVEDP. One or more of the displayed (or not displayed) metrics may be used to determine a weaning status of the patient according to the techniques described herein. Additionally, in some embodiments, one or more metrics from the patient's electronic health record (EHR) may be used to determine the weaning status of the patient. In some embodiments, information based, at least in part, on the patient's determined weaning status may be automatically stored in the patient's EHR.

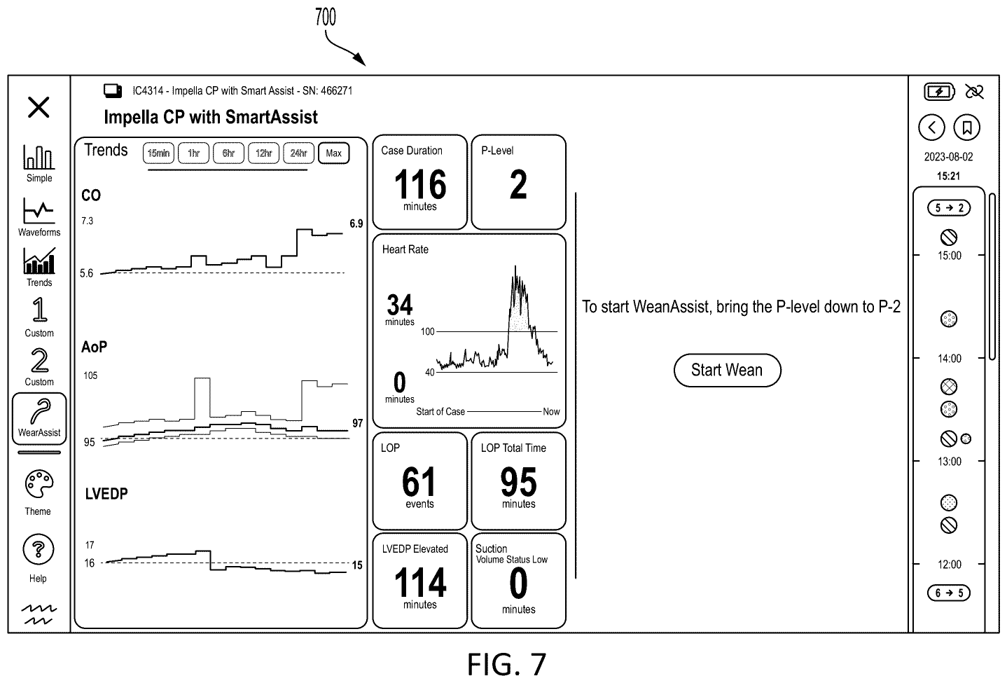

As shown in FIG. 6, user interface 600 may include instructions to start the weaning status determination process. In the example shown in FIG. 6, the user is instructed to slow the pump speed down to a lower level (e.g., a P-level of P-2) prior to starting the weaning status determination process. It should be appreciated, however, that not all embodiments of the present technology require slowing down the pump speed prior to initiating the weaning status determination. Rather, in some embodiments, the weaning status of the patient may be determined at any suitable pump speed including, but not limited to, the pump speed at which the pump is operating when the indication to initiate the weaning status determination is received in act 510 of process 500. FIG. 7 illustrates an example of a portion of a user interface 700 configured to display a user interface element that enables a user to initiate a weaning status determination, in accordance with some embodiments of the present technology.

In some embodiments, the user interface 700 may be further configured to display one or more user interface elements (not shown) with which a healthcare provider may interact to simulate how changing the pump speed or some other operating characteristic of the pump may be tolerated by the patient. For instance, the pump implanted in a patient may be currently operating at a pump speed of P-7. In some embodiments, a healthcare provider may interact with the user interface 700 to simulate reducing the pump speed to a pump speed of P-6. In response to receiving an indication that the healthcare provider would like to perform such a simulation, a set of feature values may be provided to the trained model and a simulated weaning status indication (e.g., a weaning score) for the patient may be output. By enabling the healthcare provider to simulate how changes in operating parameters of the pump (e.g., pump speed) affect the weaning status indication, the healthcare provider may gain a better understanding of how different features contribute to the determination of the weaning status indication. Additionally, the results of the simulation may enable the healthcare provider to better estimate how a patient is likely to tolerate reductions in pump speed at one or more levels prior to initiating a weaning process and/or during a weaning process. In another example, a simulation may enable the healthcare provider to determine how the weaning status indication would change if patient health data changed in particular ways and/or if the pump speed was maintained at a certain level rather than weaning the patient. Such information may be informative to the healthcare provider when deciding on treatment options for the patient.

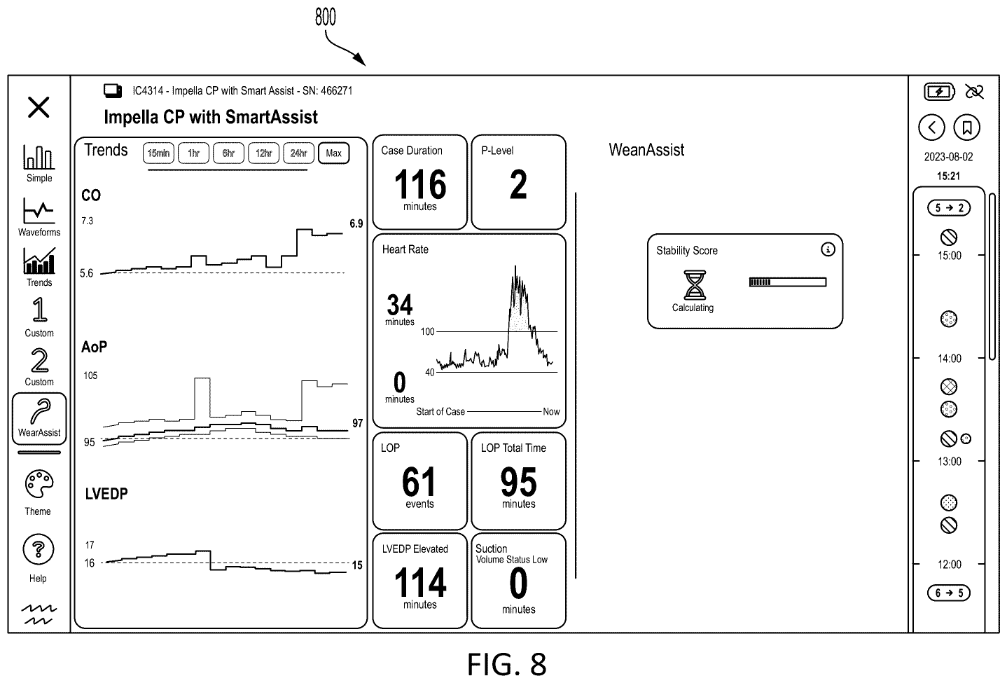

Returning to process 500, in response to receiving an indication to determine a weaning status for the patient, process 500 may proceed to act 520, where a set of feature values may be provided as input to a trained model (e.g., a machine learning model trained in accordance with the techniques described herein). For example, a user may interact with a user interface element displayed on a user interface (e.g., user interface 700), and in response, a plurality of values for features associated with the patient and/or the pump may be determined and provided as input to the trained model. In some embodiments, a same set of features used to train the model may be determined and provided as input to the trained model in act 520. In other embodiments, a subset of the set of features used to train the model (e.g., only those features most predictive of tolerance/non-tolerance) may be determined and provided as input to the trained model in act 520. In some embodiments, one or more features not used to train the model (e.g., one or more values received from the patient's EHR) may also be used to determine a weaning status for the patient. FIG. 8 illustrates an example of a portion of a user interface 800 configured to display an indication that the weaning status for the patient is being calculated based on the set of feature values provided as input to the trained model, in accordance with some embodiments of the present technology.

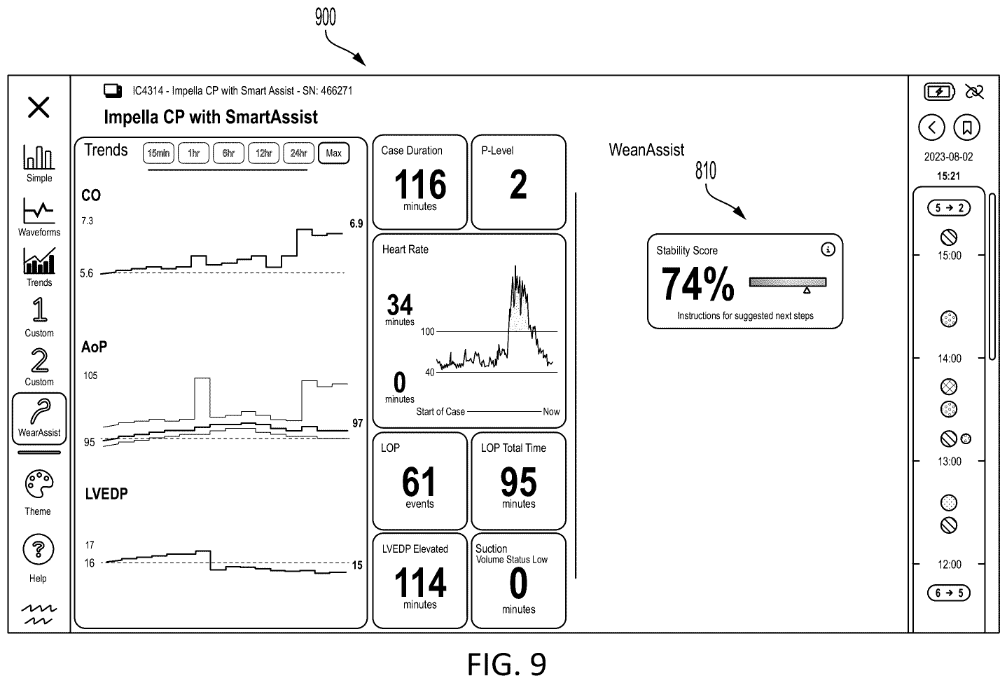

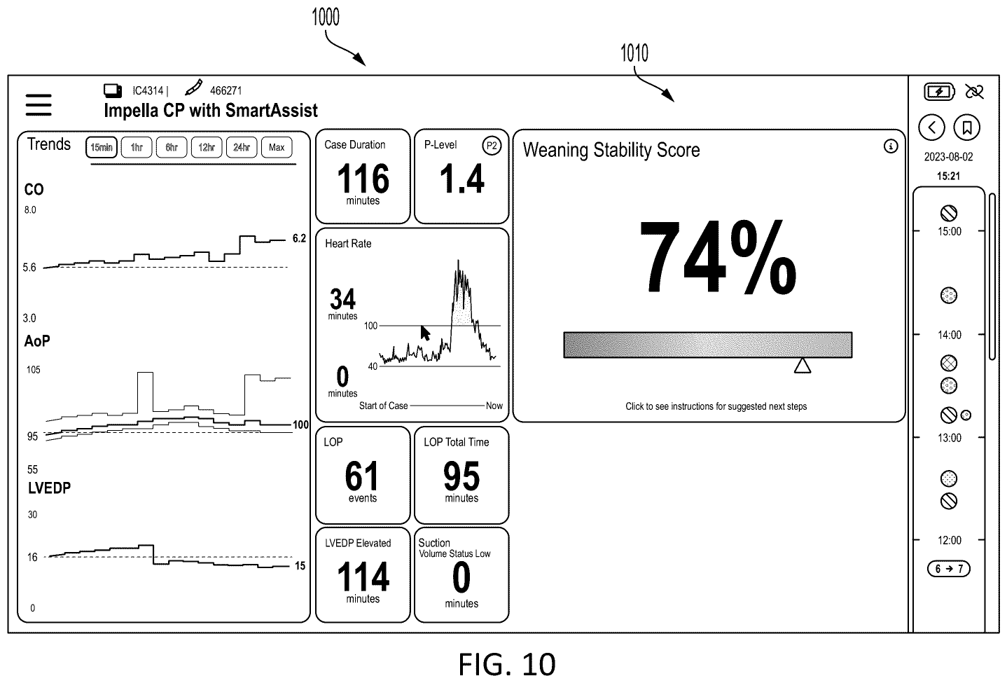

After providing the set of feature values as input to the trained model, process 500 may proceed to act 530, where an indication of the weaning status of the patient may be displayed on the user interface, wherein the indication of the weaning status is determined based, at least in part, on an output of the trained model. FIG. 9 illustrates a portion of a user interface 900 configured to display a weaning status 910 of the patient, in accordance with some embodiments of the present technology. In the example shown in FIG. 9, the weaning status 910 represents a hemodynamic stability score and includes a percentage and an associated color bar metric that may be used to determine, at least in part, whether the patient is ready to initiate a weaning process or, when determined during the weaning process, is tolerating the weaning process well or not. A healthcare provider viewing user interface 900 may use the weaning status 910 to guide decisions about whether, when and/or how to provide reductions in pump speed as the patient proceeds through the weaning process. In this way, some embodiments of the present technology may be used as a clinical decision support tool to facilitate decision making regarding weaning a patient off of a MCS device. FIG. 10 illustrates a portion of a user interface 1000 configured to display a weaning status 1010 of the patient, in accordance with some embodiments of the present technology.

In some embodiments, the user interface configured to display the indication of the weaning status (e.g., user interface 900, user interface 1000) may further be configured to display one or more user interface elements that enable a healthcare provider to provide feedback. For instance, the healthcare provider may interact with the user interface element(s) to comment on the weaning status indication (e.g., how the weaning went, whether the weaning recommendation was followed (e.g., yes or no, if no, why not, etc.)). As described herein, feedback provided by the healthcare provider may be used in some embodiments to retrain the model to refine the weaning status indications output from the model in future iterations of the weaning status determination process.

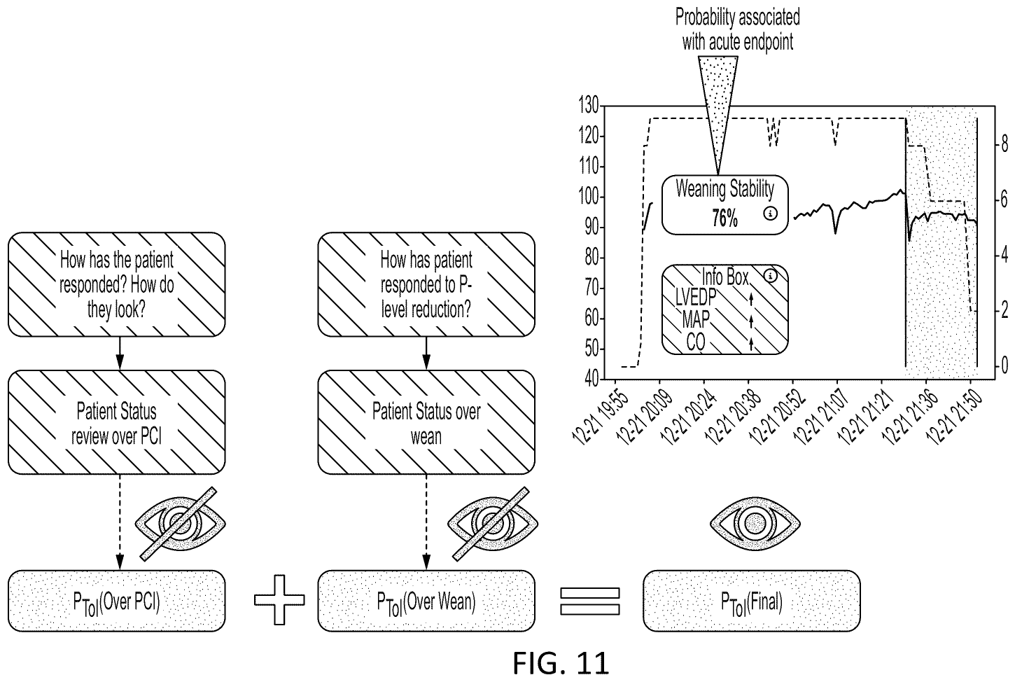

In some embodiments, the weaning status of a patient may be tracked over time. For example, the weaning status may be tracked during the PCI procedure to identify when the patient is ready to begin weaning and throughout the weaning process to determine how the patient is tolerating weaning. As shown in FIG. 11, the weaning status tracked over time may be displayed to a user and/or may be used to determine a probability or other likelihood that the patient will tolerate further reductions in MCS device support.

It should be appreciated that any of the user interfaces described herein may displayed on a display of a controller associated with the MCS device and/or may be displayed on an auxiliary display coupled to a controller of the MCS device, for example, over one or more networks.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

1. A computer-implemented method, comprising:
receiving a set of signals from a mechanical circulatory support device implanted in a heart of a patient;
determining, using a computer processor, a set of features based, at least in part, on the set of signals;
providing the set of features as input to a machine learning model trained to output a weaning status for the patient; and
displaying, on a user interface associated with the mechanical circulatory support device, an indication of the weaning status for the patient output from the machine learning model.

2. The computer-implemented method of claim 1, wherein the set of signals include at least one first signal associated with operation of the mechanical circulatory support device and at least one second signal associated with a physiology of the patient.

3. The computer-implemented method of claim 1, wherein determining a set of features based, at least in part on the set of signals comprises determining one or more of a contractility feature, a pulsatility feature, or a heart rate feature.

4. The computer-implemented method of claim 1, further comprising:
receiving, via the user interface, an indication to determine the weaning status of the patient,
wherein providing the set of features as input to a machine learning model is performed in response to receiving the indication to determine the weaning status.

5. The computer-implemented method of claim 1, further comprising:
receiving, via the user interface, user input associated with weaning the patient off the mechanical circulatory support device; and
retraining the machine learning model based, at least in part, on the user input.

6. The computer-implemented method of claim 1, further comprising:
receiving from an electronic medical record associated with the patient, medical information; and
providing the medical information as input to the machine learning model.

7. The computer-implemented method of claim 1, wherein the mechanical circulatory support device includes a heart pump, and wherein the computer-implemented method further comprises:
receiving, via the user interface, an instruction to reduce a speed of the heart pump;
sending an instruction to a controller of the heart pump to reduce the speed of the heart pump after receiving the instruction to reduce the speed; and
updating the weaning status for the patient after reducing the speed of the heart pump.

8. The computer-implemented method of claim 7, wherein updating the weaning status for the patient comprises:
determining, using the computer processor, a second set of features based, at least in part, on the set of signals received after reducing the speed of the heart pump;
providing the second set of features as input to the machine learning model; and
displaying, on the user interface associated with the mechanical circulatory support device, an indication of an updated weaning status for the patient output from the machine learning model when provided with the second set of features as input.

9. The computer-implemented method of claim 1, wherein displaying, on a user interface associated with the mechanical circulatory support device, an indication of the weaning status for the patient output from the machine learning model comprises displaying a hemodynamic stability score for the patient.

10. The computer-implemented method of claim 1, further comprising:
displaying, on the user interface, one or more user interface elements that enable a user to simulate weaning of the patient off the mechanical circulatory support device;
performing a weaning simulation in response to receiving user input via the one or more user interface elements; and
displaying, on the user interface, an updated weaning status score determined based on performing the weaning simulation.

11. A controller for a mechanical circulatory support device, the controller comprising:
at least one hardware processor configured to:
determine a set of features based, at least in part, on a set of signals received from a mechanical circulatory support device implanted in a heart of a patient;
provide the set of features as input to a machine learning model trained to output a weaning status for the patient; and
display, on a user interface associated with the mechanical circulatory support device, an indication of the weaning status for the patient output from the machine learning model.

12. The controller of claim 11, wherein the set of signals include at least one first signal associated with operation of the mechanical circulatory support device and at least one second signal associated with a physiology of the patient.

13. (canceled)

14. The controller of claim 11, wherein the at least one hardware processor is further configured to:
receive, via the user interface, an indication to determine the weaning status of the patient,
wherein providing the set of features as input to a machine learning model is performed in response to receiving the indication to determine the weaning status.

15. The controller of claim 11, wherein the at least one hardware processor is further configured to:
receive, via the user interface, user input associated with weaning the patient off the mechanical circulatory support device; and
retrain the machine learning model based, at least in part, on the user input.

16. The controller of claim 11, wherein the at least one hardware processor is further configured to:
receive from an electronic medical record associated with the patient, medical information; and
provide the medical information as input to the machine learning model.

17. The controller of claim 11, wherein the mechanical circulatory support device includes a heart pump, and wherein the at least one hardware processor is further configured to:
receive, via the user interface, an instruction to reduce a speed of the heart pump;
reduce the speed of the heart pump after receiving the instruction to reduce the speed; and
update the weaning status for the patient after reducing the speed of the heart pump.

18. The controller of claim 17, wherein updating the weaning status for the patient comprises:
determining a second set of features based, at least in part, on the set of signals received after reducing the speed of the heart pump;
providing the second set of features as input to the machine learning model; and
displaying, on the user interface associated with the mechanical circulatory support device, an indication of an updated weaning status for the patient output from the machine learning model when provided with the second set of features as input.

19. The controller of claim 11, wherein displaying, on a user interface associated with the mechanical circulatory support device, an indication of the weaning status for the patient output from the machine learning model comprises displaying a hemodynamic stability score for the patient.

20. The controller of claim 11, wherein the at least one hardware processor is further configured to:
display, on the user interface, one or more user interface elements that enable a user to simulate weaning of the patient off the mechanical circulatory support device;
perform a weaning simulation in response to receiving user input via the one or more user interface elements; and
display, on the user interface, an updated weaning status score determined based on performing the weaning simulation.

21. A heart pump system, comprising:
a heart pump including at least one pressure sensor configured to sense a pressure within a portion of a heart of a patient; and
a controller configured to:
determine a set of features based, at least in part, on a set of signals received from heart pump, the set of features a first feature based on the sensed pressure;
provide the set of features as input to a machine learning model trained to output a weaning status for the patient; and
display, on a user interface associated with the heart pump system, an indication of the weaning status for the patient output from the machine learning model.

22-31. (canceled)
